(19) United States
(12) Patent Application Publication
(10) Pub. No.: US 2025/0339166 A1
(43) Pub. Date: Nov. 6, 2025

(54) MULTIFUNCTIONAL INTEGRATED SURGICAL FORCEPS

(71) Applicant: ZHEJIANG SHUYOU SURGICAL INSTRUMENT CO.,LTD., Huzhou (CN)

(72) Inventors: Zuokai YIN, Huzhou (CN); Kongpei LU, Huzhou (CN); Kaixin ZHANG, Huzhou (CN)

(73) Assignee: ZHEJIANG SHUYOU SURGICAL INSTRUMENT CO.,LTD., Huzhou (CN)

(21) Appl. No.: 19/266,113

(22) Filed: Jul. 10, 2025

Related U.S. Application Data
(63) Continuation of application No. PCT/CN2023/130327, filed on Nov. 8, 2023.

(30) Foreign Application Priority Data
Mar. 22, 2023 (CN) 202310279410.3

(51) Int. Cl. A61B 17/29; A61B 18/00; A61B 18/08

(52) U.S. Cl. CPC A61B 17/2909; A61B 18/085; A61B 2017/292; A61B 2017/2943; A61B 2018/00589

(57) ABSTRACT

The present invention discloses a multifunctional integrated surgical forceps, comprising a multifunctional forceps head, a sleeve, a forceps head opening and closing mechanism, a housing, a first forceps head, and a second forceps head. The first forceps head has an upper toothed clamping electrocoagulation strip, and the second forceps head has a lower toothed clamping electrocoagulation strip and the coagulation strip. The tissue is clamped and electrocoagulated by the cooperation of the upper toothed clamping electrocoagulation strip and the lower toothed clamping electrocoagulation strip. The surgical process can hook, clamp, and strip the tissue. The coagulation strip effectively coagulates small blood vessels and tissue during the surgical process, making the surgical forceps have multiple functions without the need to constantly replace the surgical forceps during the surgical process, shortening the surgical time.

### RELATED APPLICATIONS

The present patent document is a continuation of PCT Application Serial No. PCT/CN2023/130327, filed Nov. 8, 2023, designating the United States and published in Chinese, which is hereby incorporated by reference.

The present patent document claims the benefit of priority to patent application Ser. No. 20/231,0279410.3, filed Mar. 22, 2023, and entitled “MULTIFUNCTIONAL INTEGRATED SURGICAL FORCEPS,” the entire contents of each of which are incorporated herein by reference.

### BACKGROUND

### 1. Technical Field

The present invention relates to a medical device, specifically a multifunctional integrated surgical forceps.

### 2. Background Information

Laparoscopy is a newly developed minimally invasive surgical method and an inevitable trend in the development of surgery in the 21st century. At present, laparoscopy can be used in most gynecological surgical treatments, such as uterine fibroids, ovarian tumors, cervical cancer, endometrial cancer, ovarian cancer, endometriosis, etc. Endoscopic abdominal surgery usually involves opening several small holes in the abdomen and inserting a puncture needle. Surgical instruments such as laparoscopy and abdominal surgical forceps are inserted into the abdominal cavity through the puncture needle. Doctors observe the situation inside the abdominal cavity through laparoscopy and use abdominal surgical forceps to clamp, grasp, pull, fix, and peel off tissues and organs during the surgery. However, the existing abdominal surgical forceps often have a single function and can only perform single clamping or peeling cutting functions, which leads to doctors needing to constantly replace the surgical forceps during laparoscopic surgery, prolonging the operation time and causing greater pain to patients.

### BRIEF SUMMARY

The purpose of the present invention is to provide a multifunctional integrated surgical forceps to solve the problems in the background technology.

The above technical objectives of the present invention are achieved through the following technical solutions:

A multifunctional integrated surgical forceps, it comprises a multifunctional forceps head, a sleeve, a forceps head opening and closing mechanism, and a housing, the sleeve is connected to the housing, and the multifunctional forceps head is set at the front end of the sleeve and extends outside the mouth of the sleeve, the forceps head opening and closing mechanism controls the opening and closing action of the multifunctional forceps head, the multifunctional forceps head comprises a first forceps head and a second forceps head, and the first forceps head is formed with an upper toothed clamping electrocoagulation strip along the length direction, and the second forceps head is formed with a lower toothed clamping electrocoagulation strip that cooperates with the upper toothed clamping electrocoagulation strip along the length direction, the inner side of the second forceps head is also provided with a coagulation strip, and the tail ends of the first forceps head and the second forceps head are both hooked.

The present invention clamps and electrocoagulates tissues through the cooperation of the upper toothed clamping electrocoagulation strip of the first forceps head and the lower toothed clamping electrocoagulation strip of the second forceps head. The tail ends of the first forceps head and the second forceps head are hooked, and the surgical process can hook, clamp, and peel off tissues. The coagulation strip effectively coagulates small blood vessels and tissues during the surgical process, making the surgical forceps have multiple functions and reducing the surgical time without constantly changing the surgical forceps during the surgery.

Preferably, the lower toothed clamping electrocoagulation strip is provided with two, and the two lower toothed clamping electrocoagulation strips are arranged parallel to each other, the lower toothed clamping electrocoagulation strip comprises several first tooth bodies and first tooth grooves arranged alternately in sequence, and the end face of the first tooth body is a first arc-shaped surface protruding upward, and the groove bottom of the first tooth groove is a second arc-shaped surface protruding downward; the upper toothed clamping electrocoagulation strip comprises several second tooth bodies and second tooth grooves arranged alternately in sequence, wherein the second tooth body corresponds to the first tooth slot, and the second tooth groove corresponds to the first tooth body.

The present invention uses the cooperation between the first tooth body and the second tooth groove, and uses the cooperation between the first tooth groove and the second tooth body to bite, thereby clamping and electrocoagulating the tissue. Through the design of the arc-shaped surface, the contact area with the tissue can be increased, making it easier to clamp.

The end face of the first tooth body and the bottom of the first tooth groove can also be flat or pointed.

Preferably, the coagulation strip is arranged between two lower toothed clamping electrocoagulation strips, the width of the coagulation strip gradually decreases from the side close to the second forceps head to the side far away from the second forceps head, and the first forceps head is provided with a V-shaped groove that cooperates with the coagulation strip.

The coagulation strip of the present invention gradually shrinks from the side close to the second forceps head to the side far away from the second forceps head, ensuring the stability and sharpness of the coagulation strip. The tissue is coagulated by the cooperation of the V-shaped groove and the coagulation strip.

Preferably, the top surface of the coagulation strip is higher than the top surface of the upper toothed clamping electrocoagulation strip, and the top surface of the V-shaped groove is higher than the inner surface of the first forceps head, so that the tissue can be coagulated after clamping.

Preferably, the forceps head opening and closing mechanism comprises a sliding rod, a sliding block, a front handle, a rear handle, and the sliding rod passes through the tube hole of the sleeve, the sliding block is fixed on the outer side of the sliding rod and is slidably connected to the sleeve, the rear handle is rotatably connected to the starting end of the sliding rod, and a second forceps head fixing plate is formed on the side of the second forceps head close to the sliding rod, the second forceps head fixing plate is sequentially provided with a first mounting hole and a second mounting hole from left to right, the height of the first mounting hole is higher than that of the second mounting hole, the sliding rod is a third mounting hole on top that cooperates with the first mounting hole, and the sliding rod and the second forceps head fixing plate are rotatably connected to the first mounting hole through the third mounting hole;

The first forceps head is formed with two first forceps head fixing plates on one side close to the sliding rod, the two first forceps head fixing plates are arranged on both sides of the second forceps head fixing plate, the first forceps head fixing plate is provided with a fourth mounting hole and a fifth mounting hole from left to right, the fourth mounting hole corresponds to the second mounting hole, and the height of the fifth mounting hole is higher than that of the fourth mounting hole, the first forceps head fixing plate and the second forceps head fixing plate is rotatably connected to the second mounting hole through the fourth mounting hole, and the fifth mounting hole is provided with a fixing pin, the inner side of the end of the sleeve is provided with an extension tube, the extension tube is provided with extension plates on both sides of the first forceps head fixing plate, and the extension plates are connected to the fixing pins.

The present invention controls the sliding rod to retract inward by pressing the rear handle, thereby closing the first forceps head and the second forceps head, completing the clamping, electrocoagulation, and coagulation functions. When the rear handle is released, the sliding rod extends outward, and the first forceps head and the second forceps head open.

Preferably, the sliding rod comprises an outer cylinder and an inner rod arranged inside the outer cylinder, the inner rod is rotatably connected to the second forceps head, the sliding block is arranged on the outer side of the outer cylinder, and a sliding plate is fixedly connected to one end of the inner rod near the outer cylinder, the upper and lower sides of the sliding plate are provided with a first gear, and the outer cylinder is provided with a gear that meshes with the first gear, the outer cylinder is equipped with a second rack that meshes with the gear, and the first rack is also equipped with a telescopic trigger switch.

The present invention controls the extension and contraction of the inner rod on the outer cylinder through the cooperation of gears and the first rack and the second rack, thereby enabling the surgical forceps to adjust their length, facilitating surgery on deeper or shallower parts, and also facilitating doctors to control their length, making the surgical forceps no longer fixed in length and easier for doctors to hold.

Preferably, the extension tube is provided with several length adjustment holes, the sleeve is provided with an insertion hole, and a pin is provided inside the insertion hole, the pin is inserted into the length adjustment hole, and the inner rod is provided with several fixed holes in an array, the angle between the fixed hole and the length adjustment hole is the same.

When the plug of the present invention is inserted into the length adjustment hole, the extension tube is fixed to the sleeve, so that the sliding rod can control the opening and closing of the clamp head. After the plug is inserted into the fixed hole, the extension tube separates from the sleeve, so that the extension tube can follow the inner rod to expand and contract, thereby completing the expansion and contraction action.

Preferably, the telescopic trigger switch comprises a support block, a compression spring, a slider, a pressing block, a swing arm, and a clamping column, the support block is fixed on the side wall of the outer cylinder, and an installation groove is opened on the support block, the slider is set in the installation groove, and two compression springs are set at the bottom of the slider, one end of the compression spring is connected to the installation groove, the slider is fixedly connected, and the other end is fixedly connected to the groove bottom of the installation groove, the side wall of the slider is provided with a heart-shaped automatic guide slot, and the swing arm one end is located in the heart-shaped automatic guidance slot, and the other end is rotatably connected to the support block, the pressing block is located at the top of the slider, and the card post is located at the bottom of the slider.

When the present invention does not require the activation of the telescopic function, the compression spring is contracted by pressing the second slider with the pressing block, and at the same time, the swing arm slides to the top of the heart-shaped automatic guide card slot, so that the bottom clamping column extends to the bottom of the support block to clamp the first rack, preventing the gear from moving without external force and affecting the surgery. When the telescopic function needs to be activated, the pressing block is pressed again, and the swing arm moves to the bottom of the heart-shaped automatic guide card slot, so that the clamping column retracts into the installation groove, thereby activating the telescopic function.

Preferably, the bottom of the clamping column is formed with a cone block, which can be easily inserted into the tooth groove of the first rack.

Preferably, a through groove is provided at the position of the telescopic trigger switch corresponding to the sleeve, and a cover body is closed on the through groove.

The cover can hide the telescopic trigger switch inside the sleeve when the telescopic function is not needed.

In summary, the beneficial effects of the present invention are:

1. The present invention clamps and electrocoagulates tissues through the cooperation of the upper toothed clamping electrocoagulation strip of the first forceps head and the lower toothed clamping electrocoagulation strip of the second forceps head. The tail ends of the first forceps head and the second forceps head are both hooked, and the surgical process can hook, clamp, and peel off tissues. The coagulation strip effectively coagulates small blood vessels and tissues during the surgical process, making the surgical forceps have multiple functions without the need to constantly replace the surgical forceps, thereby shortening the surgical time.

2. The present invention controls the extension and contraction of the inner rod on the outer cylinder through the cooperation of gears and the first rack and the second rack, thereby enabling the surgical forceps to adjust their length, facilitating surgery on deeper or shallower parts, and also facilitating doctors to control their length, making the surgical forceps no longer fixed in length and easier for doctors to hold.

3. When the plug of the present invention is inserted into the length adjustment hole, the extension tube is fixed to the sleeve, so that the sliding rod can control the opening and closing of the clamp head. After the plug is inserted into the fixed hole, the extension tube separates from the sleeve, so that the extension tube can follow the inner rod to expand and contract, thereby completing the expansion and contraction action.

4. When the present invention does not require the activation of the telescopic function, the compression spring contracts by pressing the second slider with the pressing block, and at the same time, the swing arm slides to the top of the heart-shaped automatic guide card slot, so that the bottom clamping column extends to the bottom of the support block to clamp the first rack, preventing the gear from moving without external force and affecting the surgery. When the telescopic function needs to be activated, press the pressing block again, and the swing arm moves to the bottom of the heart-shaped automatic guide card slot, so that the clamping column retracts into the installation groove, thereby activating the telescopic function.

### BRIEF DESCRIPTION OF THE DRAWINGS

FIG. 1 is a schematic diagram of the surgical forceps of the present invention;

FIG. 2 is a schematic sectional view of the front end of the surgical forceps of the present invention;

FIG. 3 is a schematic sectional view of the inner rod extending from the outer cylinder of the present invention;

FIG. 4 is a schematic diagram of the connection between the multifunctional pliers head and the extension plate of the present invention;

FIG. 5 is a schematic sectional view of the multifunctional pliers head of the present invention;

FIG. 6 is a schematic diagram of the second forceps head of the present invention;

FIG. 7 is a schematic diagram of the first forceps head of the present invention;

FIG. 8 is a schematic diagram of the multifunctional pliers head of the present invention;

FIG. 9 is a schematic sectional view of the telescopic trigger switch of the present invention;

FIG. 10 is a schematic diagram of the clamping column of the telescopic trigger switch of the present invention hidden inside the support block;

FIG. 11 is a schematic diagram of the preparation state of the multifunctional pliers head of the present invention.

The following specific embodiments are only for the explanation of the present invention and do not limit the present invention. After reading this specification, those skilled in the art may make modifications to the embodiments as needed without creative contributions, but as long as they are within the scope of the claims of the present invention, they are protected by the Patent Law.

Below, the present invention will be described in detail through examples with reference to the accompanying drawings.

### DETAILED DESCRIPTION OF THE DRAWINGS AND THE PRESENTLY PREFERRED EMBODIMENTS

### Example 1

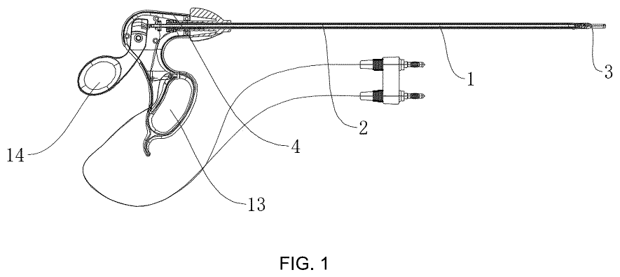

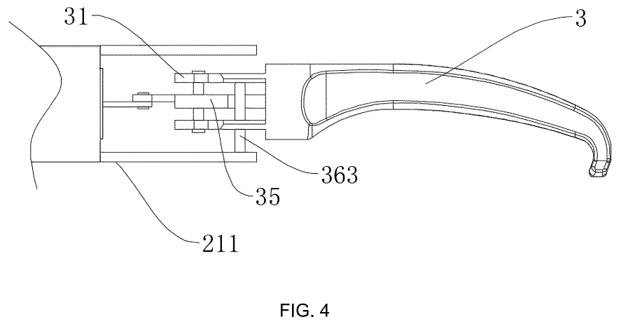

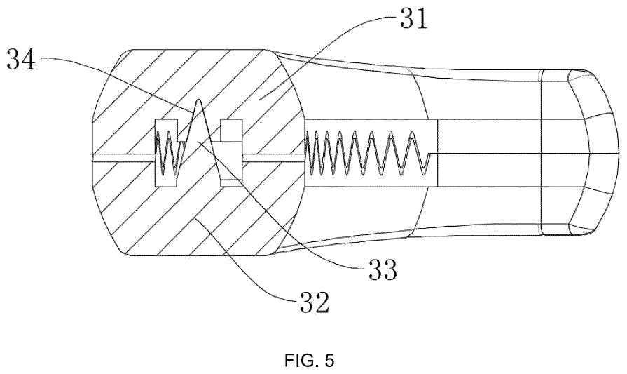

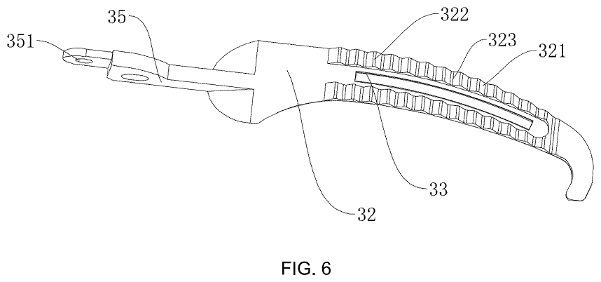

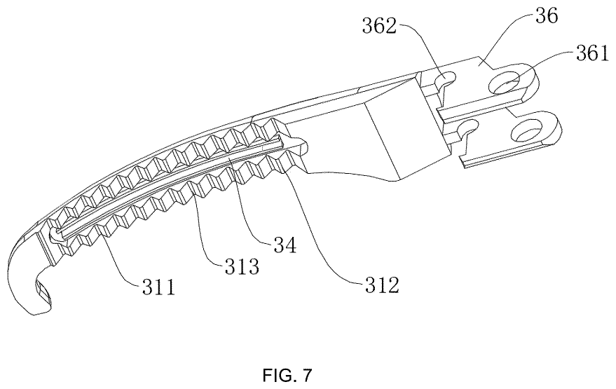

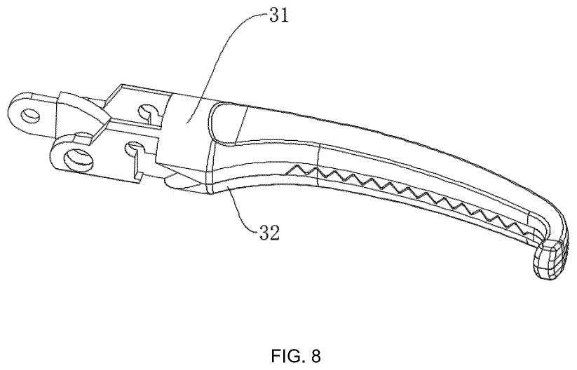

As shown in FIG. 1 and FIG. 4 to FIG. 8, a multifunctional integrated surgical forceps comprises a multifunctional forceps head 3, a sleeve 2, a forceps head opening and closing mechanism 1, and a housing 4. The sleeve 2 is connected to the housing 4, and the multifunctional forceps head 3 is set at the front end of the sleeve 2 and extends outside the mouth of the sleeve 2. The forceps head opening and closing mechanism 3 controls the opening and closing action of the multifunctional forceps head 3. The multifunctional forceps head 3 comprises a first forceps head 31 and a second forceps head 32. The first forceps head 31 is formed along the length direction with an upper toothed clamping electrocoagulation strip 311, and the second forceps head 32 is formed along the length direction with an upper toothed clamping electrocoagulation strip 311. The lower toothed clamping electrocoagulation strip 321, which cooperates with the upper toothed clamping electrocoagulation strip 311, is further equipped with a coagulation strip 33 on the inner side of the second forceps head 32. The tail ends of the first forceps head 31 and the second forceps head 32 are both hooked, and the lower toothed clamping electrocoagulation strip 321 is equipped with two, Two lower toothed clamping electrocoagulation strips 321 are arranged parallel to each other. The lower toothed clamping electrocoagulation strip 321 includes several first tooth bodies 322 and first tooth grooves 323 arranged alternately in sequence. The end face of the first tooth body 322 is a first arc-shaped surface 324 protruding upward, and the groove bottom of the first tooth groove 323 is a second arc-shaped surface 325 protruding downward; The upper toothed clamping electrocoagulation strip 311 includes several second tooth bodies 312 and second tooth grooves 313 arranged alternately in sequence. The second tooth body 312 corresponds to the first tooth groove 323, and the second tooth groove 313 corresponds to the first tooth body 323. The electrocoagulation strip 33 is located between two lower toothed clamping electrocoagulation strips 321, and the width of the electrocoagulation strip 33 gradually decreases from the side close to the second forceps head 31 to the side far away from the second forceps head 31. The first forceps head 31 is provided with a V-shaped groove 34 that cooperates with the electrocoagulation strip 33, and the top surface of the electrocoagulation strip 33 is higher than that of the electrocoagulation strip 33. The top surface of the toothed clamping electrocoagulation strip 321 is V The top surface of the slot 34 is higher than the inner surface of the first forceps head 31. The clamp head opening and closing mechanism 1 includes a sliding rod 11, a sliding block 12, a front handle 13, a rear handle 14, and a pipe hole through which the sliding rod 11 passes through the sleeve 2. The sliding block 12 is fixed on the outer side of the sliding rod 11 and is slidably connected to the sleeve 2. The rear handle 14 is rotatably connected to the starting end of the sliding rod 11. A second forceps head fixing plate 35 is formed on one side of the second forceps head 32 near the sliding rod 11. The second forceps head fixing plate 35 is sequentially provided with a first mounting hole 351 and a second mounting hole 352 from left to right. The height of the installation hole 351 is higher than the height of the second installation hole 352, and the sliding rod 11 is provided with a third installation hole 110 that cooperates with the first installation hole, The sliding rod 11 is rotatably connected to the second forceps head fixing plate 35 through the third mounting hole 110 and the first mounting hole 351; The first forceps head 31 is formed with two first forceps head fixing plates 36 on one side close to the sliding rod 11. The two first forceps head fixing plates 36 are arranged on both sides of the second forceps head fixing plate 35. The first forceps head fixing plate 36 is provided with a fourth mounting hole 361 and a fifth mounting hole 362 from left to right, and the fourth mounting hole 361 corresponds to the second mounting hole 362. The height of the fifth mounting hole 362 is higher than that of the fourth mounting hole 361. The first forceps head fixing plate 36 and the second forceps head fixing plate 35 are rotatably connected to the second mounting hole 361 through the fourth mounting hole 361. The fifth mounting hole 361 is connected to the second mounting hole 361. There is a fixed pin 363 on the hole 362, and an extension tube 21 is provided on the inner side of the end of the sleeve 2. The extension tube 21 is located on both sides of the first forceps head fixing plate 36 and is equipped with extension plates 211, which are connected to the fixed pin 363.

### Example 2

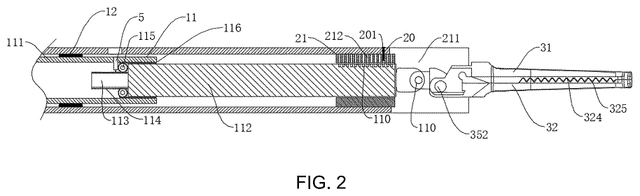

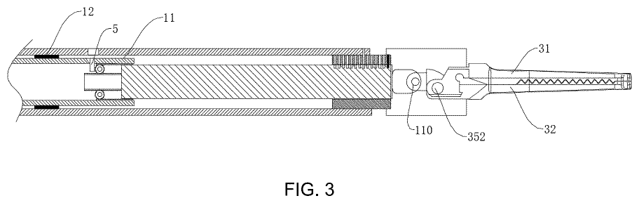

Unlike example 1, as shown in FIG. 2 to FIG. 4, the sliding rod 11 comprises an outer cylinder 111 and an inner rod 112 arranged inside the outer cylinder 111. The inner rod 112 is rotatably connected to the second forceps head 32, and the sliding block 12 is arranged on the outer side of the outer cylinder 111. One end of the inner rod 112 near the outer cylinder is fixedly connected to a sliding plate 113. The upper and lower sides of the sliding plate 113 are provided with a first rack 114, and the outer cylinder 111 is provided with a gear 115 that meshes with the first rack 114. The outer cylinder 111 is provided with a second rack 116 that meshes with the gear 115, and the first rack 114 is also provided with a telescopic trigger switch 5. The extension tube 21 is provided with several length adjustment holes 212, and the sleeve 2 is provided with an insertion hole 20. The insertion hole 20 is equipped with a pin 201, which is inserted into the length adjustment hole 212, There are several fixed holes 110 arranged in an array on the inner rod 112.

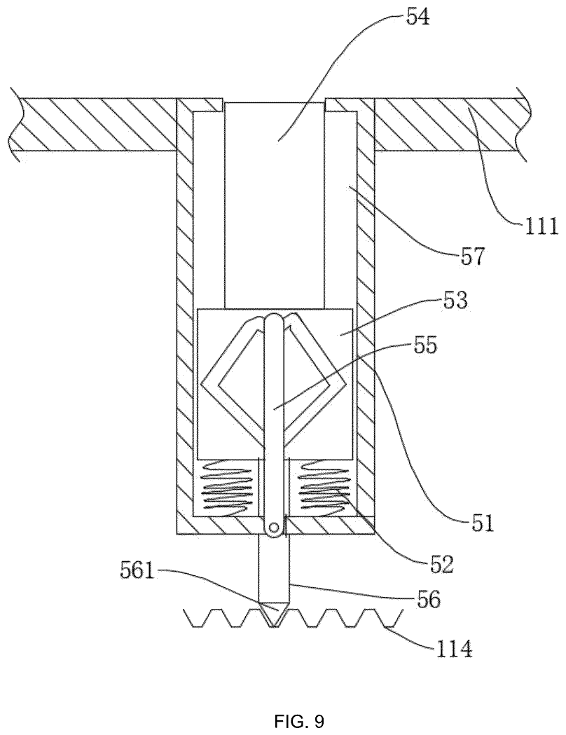

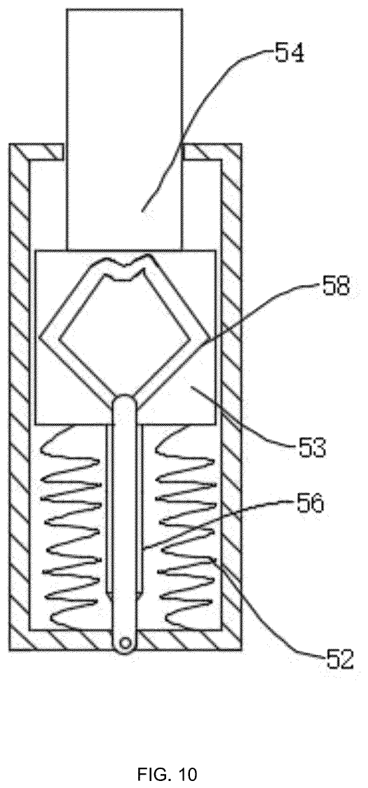

As shown in FIG. 9 to FIG. 10, the telescopic trigger switch 5 includes a support block 51, a compression spring 52, a slider 53, a pressing block 54, a swing arm 55, and a clamp column 56. The support block 51 is fixed on the side wall of the outer cylinder 111, and an installation groove 57 is opened on the support block 51. The slider 53 is set in the installation groove 57, and two compression springs 52 are set at the bottom of the slider 53. One end of the compression spring 52 is fixedly connected to the slider 53, and the other end is fixedly connected to the groove bottom of the installation groove 57. A heart-shaped automatic guidance slot 58 is opened on the side wall of the slider 53, and one end of the swing arm 55 Located in the heart-shaped automatic guidance slot 58, the other end is rotatably connected to the support block 51, the pressing block 54 is located at the top of the slider 53, the card column 56 is located at the bottom of the slider 53, and the bottom of the card column 56 is formed with a cone block 561, The sleeve 2 is provided with a through groove 51 at the position corresponding to the telescopic trigger switch 5, and a cover body 52 is closed on the through groove 51.

working principle:

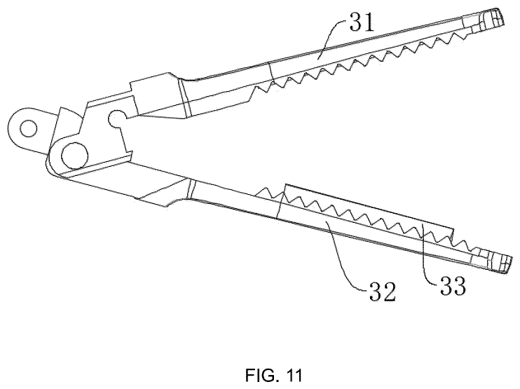

As shown in FIG. 1-11, the working principle is as follows: when adjusting the length of the surgical forceps during use, the pin 201 is pulled out from the side wall of the sleeve 20 and inserted into the extension tube 21 and the inner rod 112, so that the extension tube 21 can move along with the inner rod 112. Then, the cover 52 is opened, and the slider 53 is pressed by the pressing block 54, causing the compression spring 52 to rebound. The swing arm 55 moves to the bottom of the heart-shaped automatic guide slot 58, so that the clamp 56 is retracted into the installation slot 57. After that, the inner rod 112 is dragged, and through the cooperation of the gear 115, the first rack 114, and the second rack 116, the inner rod 112 is controlled to expand and contract on the outer tube 111. After adjusting to the appropriate length, the expansion trigger switch 5 is pressed. Block 54 presses and slides block 53, causing compression spring 52 to contract, while swing arm 55 slides to the top of the heart-shaped automatic guide slot 58, Thus, the bottom clamp 56 extends out of the support block 51 to clamp the first rack, preventing the gear 115 from rotating without external force and affecting the surgery. The pin 201 is pulled out and inserted into the insertion hole 20 of the sleeve and the length adjustment hole 212 of the extension tube 21 to fix the extension tube 21. Then, the tissue is hooked, clamped, and peeled off through the hook shape of the first forceps head 31 and the second forceps head 32. The tissue is clamped and electrocoagulated through the cooperation of the upper toothed clamping electrocoagulation strip 311 and the lower toothed clamping electrocoagulation strip 321 of the second forceps head 32. The coagulation strip 33 effectively coagulates small blood vessels and tissue during the surgery, making the surgical forceps have multiple functions without the need to clamp and electrocoagulate. Continuously changing surgical forceps during the surgery shortened the operation time.

1. A multifunctional integrated surgical forceps, characterized in that it comprises a multifunctional forceps head (3), a sleeve (2), a forceps head opening and closing mechanism (1), and a housing (4), the sleeve (2) is connected to the housing (4), and the multifunctional forceps head (3) is set at the front end of the sleeve (2) and extends outside the mouth of the sleeve (2), the forceps head opening and closing mechanism (1) controls the opening and closing action of the multifunctional forceps head (3), the multifunctional forceps head (3) comprises a first forceps head (31) and a second forceps head (32), and the first forceps head (31) is formed with an upper toothed clamping electrocoagulation strip (311) along the length direction, and the second forceps head (32) is formed with a lower toothed clamping electrocoagulation strip (321) that cooperates with the upper toothed clamping electrocoagulation strip (311) along the length direction, the inner side of the second forceps head (32) is also provided with a coagulation strip (33), and the tail ends of the first forceps head (31) and the second forceps head (32) are both hooked.

2. A multifunctional integrated surgical forceps according to claim 1, characterized in that the lower toothed clamping electrocoagulation strip (321) is provided with two, and the two lower toothed clamping electrocoagulation strips (321) are arranged parallel to each other, the lower toothed clamping electrocoagulation strip (321) comprises several first tooth bodies (322) and first tooth grooves (323) arranged alternately in sequence, and the end face of the first tooth body (322) is a first arc-shaped surface (324) protruding upward, and the groove bottom of the first tooth groove (323) is a second arc-shaped surface (325) protruding downward; the upper toothed clamping electrocoagulation strip (311) comprises several second tooth bodies (312) and second tooth grooves (313) arranged alternately in sequence, wherein the second tooth body (312) corresponds to the first tooth slot (323), and the second tooth groove (313) corresponds to the first tooth body (323).

3. A multifunctional integrated surgical forceps according to claim 2, characterized in that the coagulation strip (33) is arranged between two lower toothed clamping electrocoagulation strips (321), the width of the coagulation strip (33) gradually decreases from the side close to the second forceps head (31) to the side far away from the second forceps head (31), and the first forceps head (31) is provided with a V-shaped groove (34) that cooperates with the coagulation strip (33).

4. A multifunctional integrated surgical forceps according to claim 3, characterized in that the top surface of the coagulation strip (33) is higher than the top surface of the upper toothed clamping electrocoagulation strip (321), and the top surface of the V-shaped groove (34) is higher than the inner surface of the first forceps head (31).

5. A multifunctional integrated surgical forceps according to claim 1, characterized in that the forceps head opening and closing mechanism (1) comprises a sliding rod (11), a sliding block (12), a front handle (13), a rear handle (14), and the sliding rod (11) passes through the tube hole of the sleeve (2), the sliding block (12) is fixed on the outer side of the sliding rod (11) and is slidably connected to the sleeve (2), the rear handle (14) is rotatably connected to the starting end of the sliding rod (11), and a second forceps head fixing plate is formed on the side of the second forceps head (32) close to the sliding rod (11), the second forceps head fixing plate (35) is sequentially provided with a first mounting hole (351) and a second mounting hole (352) from left to right, the height of the first mounting hole (351) is higher than that of the second mounting hole (352), the sliding rod (11) is a third mounting hole (110) on top that cooperates with the first mounting hole, and the sliding rod (11) and the second forceps head fixing plate (35) are rotatably connected to the first mounting hole (351) through the third mounting hole (110);
the first forceps head (31) is formed with two first forceps head fixing plates (36) on one side close to the sliding rod (11), the two first forceps head fixing plates (36) are arranged on both sides of the second forceps head fixing plate (35), the first forceps head fixing plate (36) is provided with a fourth mounting hole (361) and a fifth mounting hole (362) from left to right, the fourth mounting hole (361) corresponds to the second mounting hole (362), and the height of the fifth mounting hole (362) is higher than that of the fourth mounting hole (361), the first forceps head fixing plate (36) and the second forceps head fixing plate (35) is rotatably connected to the second mounting hole (361) through the fourth mounting hole (361), and the fifth mounting hole (362) is provided with a fixing pin (363), the inner side of the end of the sleeve (2) is provided with an extension tube (21), the extension tube (21) is provided with extension plates (211) on both sides of the first forceps head fixing plate (36), and the extension plates (211) are connected to the fixing pins (363).

6. A multifunctional integrated surgical forceps according to claim 5, characterized in that the sliding rod (11) comprises an outer cylinder (111) and an inner rod (112) arranged inside the outer cylinder (111), the inner rod (112) is rotatably connected to the second forceps head (32), the sliding block (12) is arranged on the outer side of the outer cylinder (111), and a sliding plate (113) is fixedly connected to one end of the inner rod (112) near the outer cylinder, the upper and lower sides of the sliding plate (113) are provided with a first gear (114), and the outer cylinder (111) is provided with a gear (115) that meshes with the first gear (114), the outer cylinder (111) is equipped with a second rack (116) that meshes with the gear (115), and the first rack (114) is also equipped with a telescopic trigger switch (5).

7. A multifunctional integrated surgical forceps according to claim 6, characterized in that the extension tube (21) is provided with several length adjustment holes (212), the sleeve (2) is provided with an insertion hole (20), and a pin (201) is provided inside the insertion hole (20), the pin (201) is inserted into the length adjustment hole (212), and the inner rod (112) is provided with several fixed holes (110) in an array.

8. A multifunctional integrated surgical forceps according to claim 6, characterized in that the telescopic trigger switch (5) comprises a support block (51), a compression spring (52), a slider (53), a pressing block (54), a swing arm (55), and a clamping column (56), the support block (51) is fixed on the side wall of the outer cylinder (111), and an installation groove (57) is opened on the support block (51), the slider (53) is set in the installation groove (57), and two compression springs (52) are set at the bottom of the slider (53), one end of the compression spring (52) is connected to the installation groove (57), the slider (53) is fixedly connected, and the other end is fixedly connected to the groove bottom of the installation groove (57), the side wall of the slider (53) is provided with a heart-shaped automatic guide slot (58), and the swing arm (55) one end is located in the heart-shaped automatic guidance slot (58), and the other end is rotatably connected to the support block (51), the pressing block (54) is located at the top of the slider (53), and the card post (56) is located at the bottom of the slider (53).

9. A multifunctional integrated surgical forceps according to claim 6, characterized in that the bottom of the clamping column (56) is formed with a cone block (561).

10. A multifunctional integrated surgical forceps according to claim 6, characterized in that a through groove (51) is opened at the position corresponding to the telescopic trigger switch (5) of the sleeve (2), and a cover body (52) is closed on the through groove (51).
